(19) United States
(12) Patent Application Publication
(10) Pub. No.: US 2026/0199134 A1
(43) Pub. Date: Jul. 16, 2026

(54) EYE MASK AND EYE MASK SET

(71) Applicant: Dongguan City YuHuang Trading Co., Ltd., Dongguan (CN)

(72) Inventor: Yuhua JIANG, Shaoyang (CN)

(73) Assignee: Dongguan City YuHuang Trading Co., Ltd., Dongguan (CN)

(21) Appl. No.: 19/449,389

(22) Filed: Jan. 14, 2026

(30) Foreign Application Priority Data
Jan. 15, 2025 (CN) 202510064587.0

(51) Int. Cl. A61F 9/04

(52) U.S. Cl. CPC A61F 9/04

(57) ABSTRACT

An eye mask includes a mask body and an adjustment member. The mask body has an outer end surface. At least a portion of the adjustment member is arranged on a side of the outer end surface of the mask body. A first end of the adjustment member adjacent to the outer end surface of the mask body is connected to the mask body, and a second end of the adjustment member facing away from the outer end surface is configured to be connected to the head-wearable device. The adjustment member is configured to generate an extension/retraction amount along a predetermined direction OO’ when subjected to force, thereby allowing a distance between the outer end surface of the mask body and an inner surface of the head-wearable device to be adjustable. The predetermined direction OO’ is inclined relative to the outer end surface of the mask body.

### CROSS-REFERENCE TO RELATED APPLICATIONS

The present disclosure claims priority of Chinese patent application No. 202510064587.0, filed on January 15, 2025, the entire contents of which are incorporated herein by reference.

### TECHNICAL FIELD

The present disclosure relates to daily necessities, and in particular, to an eye mask and an eye mask set.

### BACKGROUND

Eye masks are designed to block out light and are suitable for wear during postoperative recovery. It is understandable that users have different facial bone structures—some have higher cheekbones and foreheads, while others have lower ones. Due to uniform structures of conventional eye masks, when users with different facial features wear the eye masks, fit varies significantly. For instance, an eye mask may fit comfortably for someone with lower cheekbones and forehead but feel too tight for those with higher ones. As a result, applicability of such eye masks is limited, catering only to a narrow range of users.

### SUMMARY

In order to solve shortcomings in existing technologies, the present disclosure is to provide an eye mask and an eye mask set to solve above technological problems.

A first aspect of the present disclosure provides an eye mask configured to be connected to a head-wearable device. The eye mask includes a mask body and an adjustment member. The mask body has an outer end surface. At least a portion of the adjustment member is arranged on a side of the outer end surface of the mask body. A first end of the adjustment member adjacent to the outer end surface of the mask body is connected to the mask body, and a second end of the adjustment member facing away from the outer end surface is configured to be connected to the head-wearable device. The adjustment member is configured to generate an extension/retraction amount along a predetermined direction OO’ when subjected to force, thereby allowing a distance between the outer end surface of the mask body and an inner surface of the head-wearable device to be adjustable. The predetermined direction OO’ is inclined relative to the outer end surface of the mask body.

A second aspect of the present disclosure provides an eye set including a head-wearable device and an eye mask. The eye mask includes a mask body and an adjustment member. The mask body has an outer end surface, at least a portion of the adjustment member is arranged on a side of the outer end surface of the mask body. A first end of the adjustment member adjacent to the outer end surface of the mask body is connected to the mask body, and a second end of the adjustment member facing away from the outer end surface is configured to be connected to the head-wearable device. The adjustment member is configured to generate an extension/retraction amount along a predetermined direction OO’ when subjected to force, thereby allowing a distance between the outer end surface of the mask body and an inner surface of the head-wearable device to be adjustable. The predetermined direction OO’ is inclined relative to the outer end surface of the mask body.

A third aspect of the present disclosure provides an eye mask including eye mask configured to be connected to a head-wearable device. The eye mask includes a mask body having an outer end surface, an adjustment member, and a magnetic attraction structure. The adjustment member is at least partially arranged on a side of the mask body where the outer end surface is located, and a first end of the adjustment member adjacent to the outer end surface is connected to the mask body. The magnetic attraction structure is connected to a second end of the adjustment member facing away from the outer end surface. The magnetic attraction structure is configured to be connected to the head-wearable device via magnetic attraction to position the eye mask on the head-wearable device.

Based on the eye mask and eye mask set of the present disclosure, by configuring the adjustment member, the adjustment member is configured to generate the extension or contraction amount along the preset direction when force is applied, thereby changing the distance between the outer end surface of the mask body and the inner surface of the head-wearable device. Thus, when the eye mask is in a worn state, the adjustment member can be subjected to compressive forces from faces of different users, producing varying amounts of extension or contraction along the preset direction. This enables the eye mask to achieve an optimal distance between the outer end surface of the mask body and the inner surface of the head-wearable device for different users, effectively ensuring that the mask body can completely block light around the eyes of various users and improving applicability of the eye mask.

### BRIEF DESCRIPTION OF THE DRAWINGS

FIG. 1 is a schematic diagram of an eye mask according to a first embodiment of the present disclosure.

FIG. 2 is a schematic diagram of an adjustment member of the eye mask according to the first embodiment of the present disclosure.

FIG. 3 is a cross-sectional view of the adjustment member of the eye mask according to the first embodiment of the present disclosure.

FIG. 4 is a schematic diagram of the eye mask and a first pair of eyeglasses according to the first embodiment of the present disclosure.

FIG. 5 is a schematic diagram of the eye mask and a first lens of the first pair of eyeglasses according to an embodiment of the present disclosure.

FIG. 6 is a schematic diagram of the eye mask in a first usage state according to an embodiment of the present disclosure.

FIG. 7 is a schematic diagram of the eye mask including a reinforcement piece according to an embodiment of the present disclosure.

FIG. 8 is a schematic diagram of an eye mask according to a second embodiment of the present disclosure.

FIG. 9 is an exploded view of the eye mask in FIG. 8.

FIG. 10 is a schematic diagram of the eye mask and a second pair of eyeglasses according to the second embodiment of the present disclosure.

FIG. 11 is a schematic diagram of the eye mask in a second usage state according to the second embodiment of the present disclosure.

FIG. 12 is a schematic diagram of the eye mask according to the second embodiment of the present disclosure from another perspective.

FIG. 13 is a first schematic view of an eye mask according to a third embodiment of the present disclosure.

FIG. 14 is a schematic view of the eye mask in FIG. 13 from another perspective.

FIG. 15 is a second schematic view of the eye mask according to the third embodiment of the present disclosure.

FIG. 16 is a schematic view of the eye mask in a third usage state according to an embodiment of the present disclosure.

FIG. 17 is an exploded view of the eye mask in FIG. 16.

FIG. 18 is an exploded view of the eye mask in FIG. 16 from another perspective.

FIG. 19 is a schematic view of an eye mask set according to an embodiment of the present disclosure.

FIG. 20 is a schematic view of an eye mask set according to another embodiment of the present disclosure.

FIG. 21 is a schematic view of an eye mask according to another embodiment of the present disclosure.

FIG. 22 is a schematic view of an eye mask in FIG. 21 from another perspective.

FIG. 23 is a schematic view illustrating the eye mask in FIG. 21 mounted on a head-wearable device.

### DETAILED DESCRIPTION OF THE EMBODIMENTS

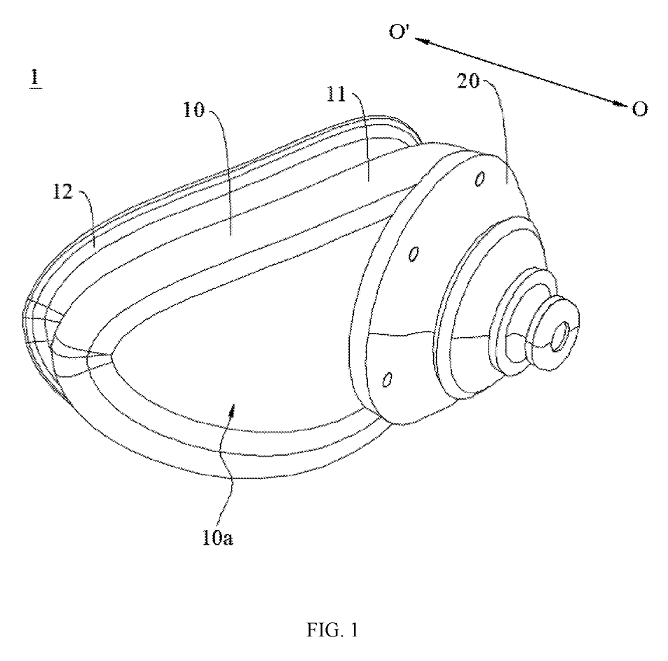

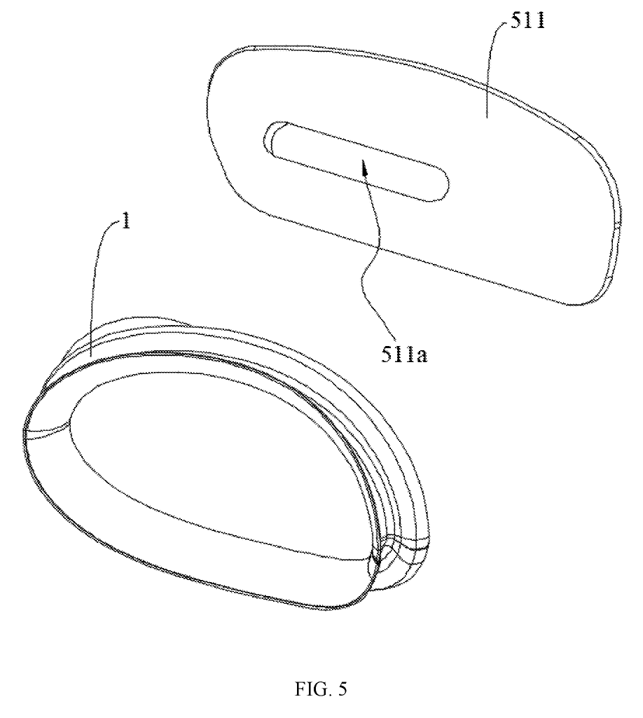

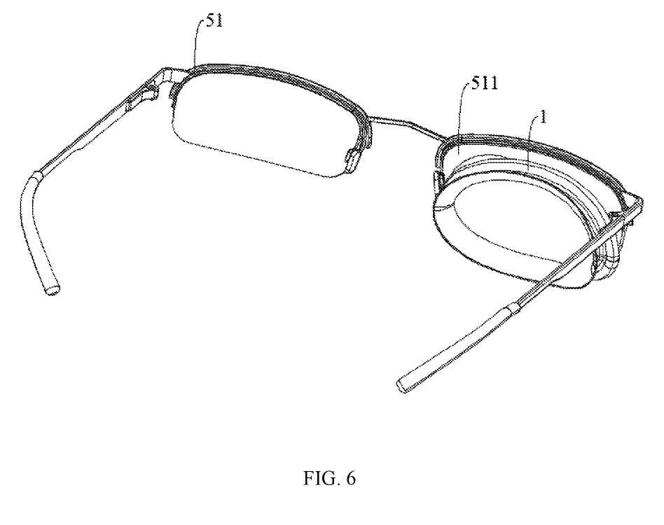

Please refer to FIGS. 1-6, Referring to FIGS. 1 to 5, in a first aspect, an embodiment of the present disclosure provides an eye mask 1, which can achieve complete shading around eyes for different users and offers wide applicability.

The eye mask 1 is configured to be connected to a head-wearable device and includes a mask body 10 and an adjustment member 20. The mask body 10 has an outer end surface 10a. At least a portion of the adjustment member 20 is arranged on a side of the outer end surface 10a of the mask body 10. A first end of the adjustment member 20 adjacent to the outer end surface 10a of the mask body 10 is connected to the mask body 10, and a second end of the adjustment member 20 facing away from the outer end surface 10a is configured to be connected to the head-wearable device. The adjustment member 20 is configured to generate an extension/retraction amount along a predetermined direction OO’ when subjected to force, thereby allowing a distance between the outer end surface 10a of the mask body 10 and an inner surface of the head-wearable device to be adjustable. The predetermined direction OO’ is inclined relative to the outer end surface 10a of the mask body 10. It can be understood that "inclined" means there is an angle between the predetermined direction OO’ and the outer end surface 10a of the mask body 10. The angle is not zero and may be 90 degrees, an acute angle, or an obtuse angle.

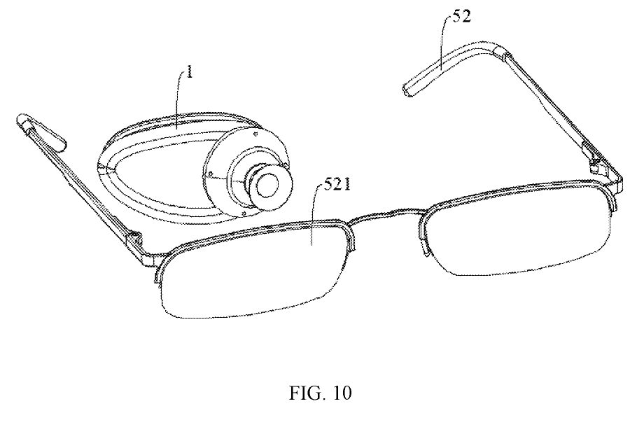

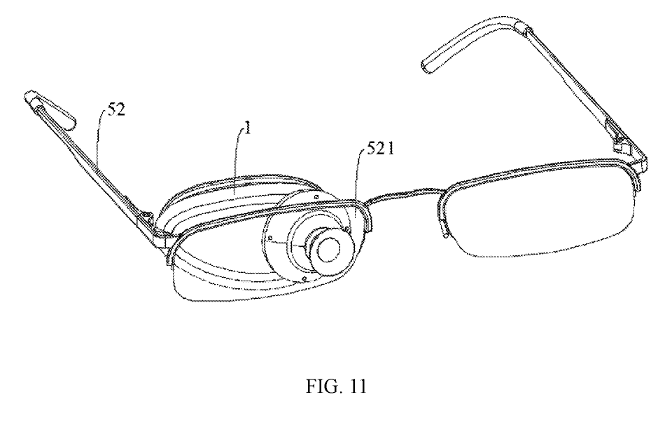

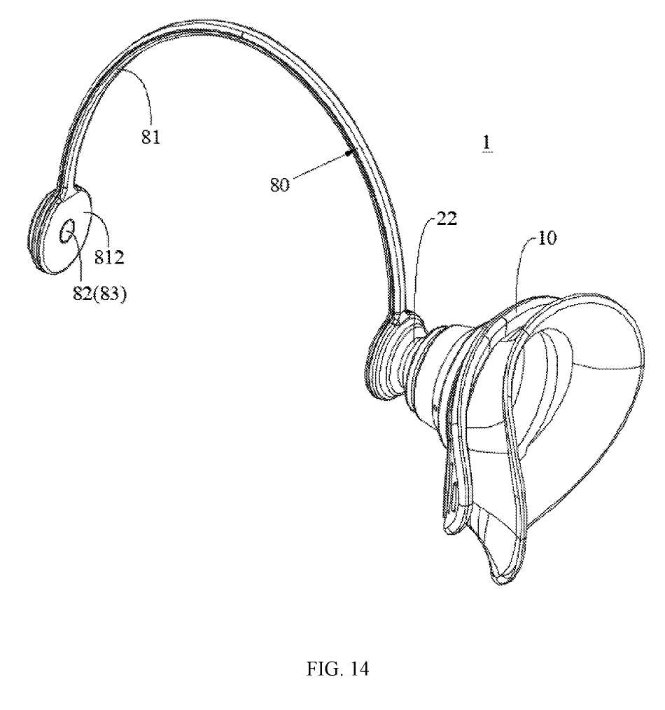

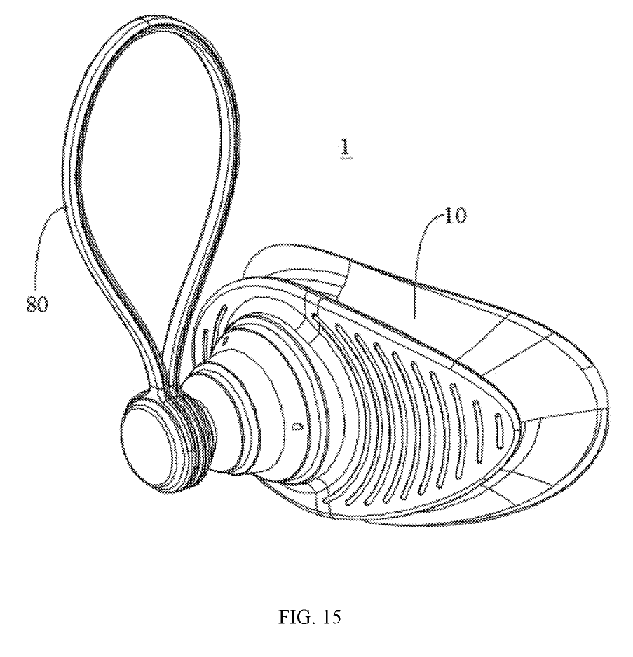

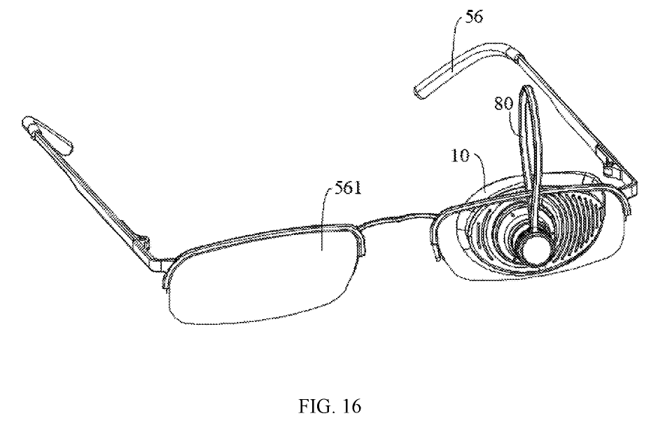

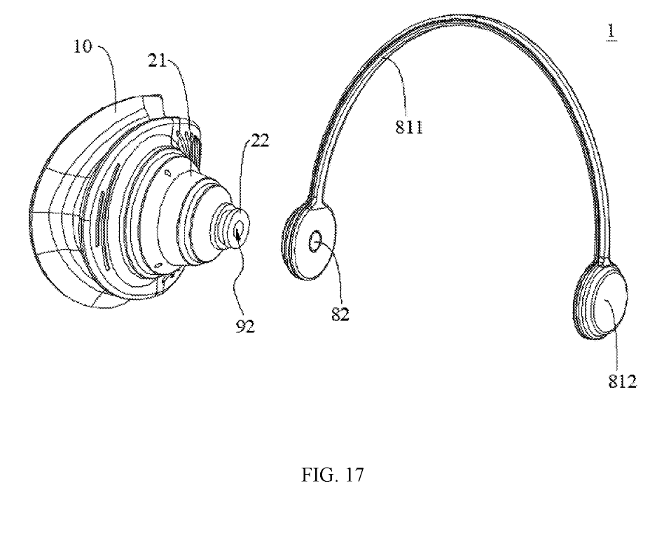

The specific structure of the eye mask 1 will now be described in detail with reference to FIGS. 1 to 18. The eye mask 1 is suitable for connection with a head-wearable device.

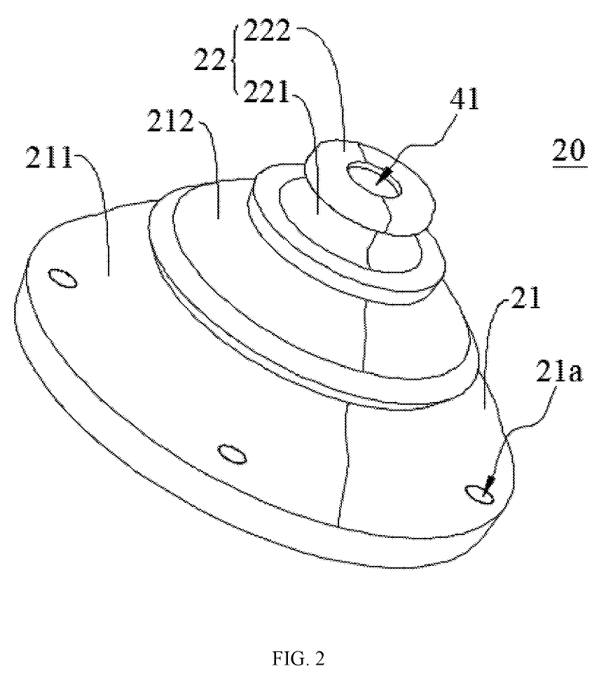

As shown in FIGS. 1 and 2, the eye mask 1 includes a mask body 10 and an adjustment member 20.

The mask body 10 functions as a shading structure of the eye mask 1. When the eye mask 1 is in a worn state, the mask body 10 covers user's eyes. The detailed structure of the mask body 10 will be further explained below. The mask body 10 has an outer end surface 10a, which may be a flat surface, a curved surface, or a combination of flat and curved surfaces.

The adjustment member 20 functions as an adjustment structure of the eye mask 1 and is configured to adjust the distance between the outer end surface 10a of the mask body 10 and the inner surface of the head-wearable device. For example, when the head-wearable device includes eyeglasses, the outer end surface 10a of the mask body 10 faces the lenses of the eyeglasses, and the adjustment member 20 can be used to adjust the distance between the outer end surface 10a and the inner surface of the lenses. The specific structure of the adjustment member 20 will be further explained below.

At least a portion of the adjustment member 20 is located on the side of the outer end surface 10a of the mask body 10. For instance, a part of the adjustment member 20 may be located on the side of the outer end surface 10a, while the remaining part is located on a side of an outer peripheral surface that is inclined relative to the outer end surface 10a. Alternatively, the entire adjustment member 20 may be located on the side of the outer end surface 10a.

The first end of the adjustment member 20 adjacent to the outer end surface 10a of the mask body 10 is connected to the mask body 10. The specific connection method between the adjustment member 20 and the mask body 10 is not limited here, and reasonable configurations can be selected according to actual needs. For example, the adjustment member 20 may be non-removably connected to the mask body 10 through methods such as bonding, injection molding, or 3D printing. Alternatively, the adjustment member 20 may be removably connected to the mask body 10 through at least one method such as screwing, snapping, or plugging.

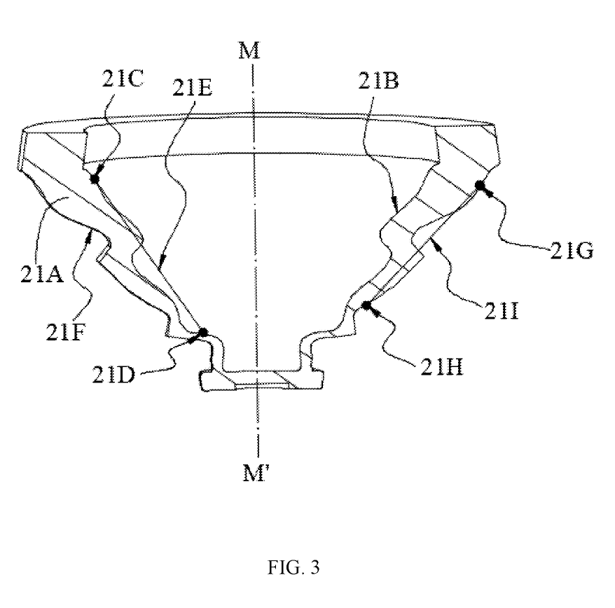

As shown in FIGS. 3 to 6, the second end of the adjustment member 20 facing away from the outer end surface 10a of the mask body 10 is configured to be connected to the head-wearable device. The specific connection method between the adjustment member 20 and the head-wearable device is not limited here, and reasonable configurations can be selected according to actual needs. For example, the adjustment member 20 may be non-removably connected to the head-wearable device through methods such as bonding, injection molding, or 3D printing. Alternatively, the adjustment member 20 may be removably connected to the head-wearable device through methods such as snapping, plugging, negative pressure adsorption, or magnetic attraction.

The adjustment member 20 is configured to generate an extension/retraction amount along a predetermined direction OO’ when subjected to force, thereby allowing the distance between the outer end surface 10a of the mask body 10 and the inner surface of the head-wearable device to be adjustable. The predetermined direction OO’ is inclined relative to the outer end surface 10a of the mask body 10 (i.e., the predetermined direction OO’ includes at least a component perpendicular to the outer end surface 10a of the mask body 10). Taking a head-wearable device including eyeglasses as an example, it is understandable that different users have different facial bone structures. For instance, for users with higher cheekbones and foreheads, when the eye mask 1 is in a worn state, the adjustment member 20 is subjected to compressive force from the user's face, generating a larger extension/retraction amount along the predetermined direction OO’. In this case, the distance between the outer end surface 10a of the mask body 10 and the inner surface of the lenses is smaller, and the mask body 10 does not exert excessive compressive force on the periphery of the user's eyes. This ensures that the mask body 10 can completely block light around the user's eyes while enhancing wearing comfort. On the other hand, for users with lower cheekbones and foreheads, when the eye mask 1 is in a worn state, the adjustment member 20 is subjected to compressive force from the user's face, generating a smaller extension/retraction amount along the predetermined direction OO’. In this case, the distance between the outer end surface 10a of the mask body 10 and the inner surface of the lenses is larger, and the mask body 10 similarly does not exert excessive compressive force on the periphery of the user's eyes. This ensures that the mask body 10 can completely block light around the user's eyes while enhancing wearing comfort.

Based on the eye mask 1 in the embodiments of the present disclosure, by configuring the adjustment member 20 to generate the extension/retraction amount along the predetermined direction OO’ when subjected to force, the distance between the outer end surface 10a of the mask body 10 and the inner surface of the head-wearable device can be adjusted. Thus, when the eye mask 1 is in a worn state, the adjustment member 20 can be subjected to compressive forces from different users' faces, generating varying extension/retraction amounts along the predetermined direction OO’. This allows the distance between the outer end surface 10a of the mask body 10 and the inner surface of the head-wearable device to be optimized for different users. As a result, it effectively ensures that the mask body 10 can completely block light around the eyes of various users, improves the applicability of the eye mask 1, and simultaneously enhances user comfort during wear.

As shown in FIGS. 1 and 2, the adjustment member 20 includes an adjustment cylinder 21 configured to generate the extension/retraction amount along the predetermined direction OO’. One end of the adjustment cylinder 21 adjacent to the outer end surface 10a of the mask body 10 serves as the first end of the adjustment member 20 and is connected to the mask body 10.

In at least one embodiment, from the side adjacent to the outer end surface 10a of the mask body 10 to the side farther away from the outer end surface 10a, the diameter of at least a portion of the adjustment cylinder 21 gradually decreases. For example, the diameter of the portion of the adjustment cylinder 21 adjacent to the outer end surface 10a of the mask body 10 may exhibit a gradually decreasing trend, while the diameter of the remaining portion farther away from the outer end surface 10a may remain unchanged. Alternatively, the diameter of the entire adjustment cylinder 21 may exhibit a gradually decreasing trend.

In at least one embodiment, alternatively, from the side adjacent to the outer end surface 10a of the mask body 10 to the side farther away from the outer end surface 10a of the mask body 10, the diameter of at least a portion of the adjustment cylinder 21 gradually increases. For example, the diameter of the portion of the adjustment cylinder 21 adjacent to the outer end surface 10a of the mask body 10 may exhibit a gradually increasing trend, while the diameter of the remaining portion farther away from the outer end surface 10a may remain unchanged. Alternatively, the diameter of the entire adjustment cylinder 21 may exhibit a gradually increasing trend.

By configuring the adjustment cylinder 21 with varying diameters along at least a portion of its length, the adjustment cylinder 21 can more easily generate an extension/retraction amount along the predetermined direction OO’ under compressive force. This allows the distance between the outer end surface 10a of the mask body 10 and the inner surface of the head-wearable device to be adjusted according to different users' facial structures, ensuring that the mask body 10 can completely block light around the user's eyes while enhancing wearing comfort.

Please referring to FIG. 3, the adjustment cylinder 21 has a thin-walled structure and extends along an axis MM' that passes through a geometric center of a cross-section of the adjustment cylinder 21. This axis MM' is substantially parallel to or coincident with the predetermined direction OO’. A cross-section 21A of the adjustment cylinder 21 is a plane where the axis MM' is located and the cross-section MM' of the adjustment cylinder 21 has an inner edge line 21B facing the axis MM'. The inner edge line 21B is formed by an inner wall of the adjustment cylinder 21. The inner edge line 21B on a side of the axis MM' has a first point 21C and a second point 21D, and a first connecting line 21E between the first point 21C and the second point 21D is inclined relative to the axis MM'. The first point 21C and the second point 21D are any two different points on the inner edge line 21B. For example, the first point 21C may be an endpoint on the inner edge line adjacent to the outer end surface 10a of the mask body 10, and the second point 21D may be an endpoint farthest from the outer end surface 10a. In this case, the first connecting line 21E connects the two endpoints of the inner edge line 21B. Alternatively, the first point 21C may be a point on the inner edge line 21B adjacent to the outer end surface 10a of the mask body 10, and the second point 21D may be a point on the inner edge line 21B farther from the outer end surface 10a. In this case, the first point 21C and the second point 21D are located between the two endpoints on the inner edge 21B and the first connecting line 21E connects the first point 21C and the second point 21D. It can be understood that the first point 21C and the second point 21D are any two different points on the inner edge line 21B, and the first connecting line 21E is inclined relative to the axis MM', meaning that the inner edge line 21B is inclined relative to the axis MM'.

The cross-section 21A of the adjustment cylinder 21 along the axis MM' has an outer edge line 21F corresponding to an outer surface of the cylinder 21. The outer edge line 21F has a third point 21G and a fourth point 21H, and a second connecting line 21I between the third point 21G and the fourth point 21H is inclined relative to the axis MM'. The third point 21G and the fourth point 21H are any two different points on the outer edge line 21F. For example, the third point 21G may be an endpoint on the outer edge line 21F adjacent to the outer end surface 10a of the mask body 10, and the fourth point 21H may be an endpoint farthest from the outer end surface 10a. In this case, the second connecting line 21I connects the two endpoints of the outer edge line 21F. Alternatively, the third point 21G may be a point on the outer edge line adjacent to the outer end surface 10a of the mask body 10, and the fourth point 21H may be a point on the outer edge line 21F farther from the outer end surface 10a. In this case, the third point 21G and the fourth point 21H are located between the two endpoints on the outer edge line 21F and the second connecting line 21I connects the third point 21G and the fourth point 21H. It can be understood that the third point 21G and the fourth point 21H are any two different points on the outer edge line 21F on a same side of the axis MM', and the second connecting line 21F is inclined relative to the axis MM', meaning the outer edge line 21F as a whole is inclined relative to the axis MM'.

Please referring to FIGS. 1 and 2, the adjustment cylinder 21 can be made of, but not limited to, flexible materials such as rubber, silicone, etc. Under force, the adjustment cylinder 21 may deflect. When the eye mask 1 is in a worn state, the adjustment cylinder 21 configured deflect in different directions can effectively ensure that the mask body 10 completely blocks light around the eyes of different users, thereby improving the applicability of the eye mask 1. It should be noted that adjustment can be achieved solely through the extension/retraction of the adjustment cylinder 21 along the predetermined direction OO’, solely through deflection of the adjustment cylinder 21, or through a combination of both.

The wall of the adjustment cylinder 21 is provided with at least one air hole 21a. The number of the at least one air hole 21a may be one or multiple. When there are multiple air holes 21a, they may be arranged at equal intervals at the wall of adjustment cylinder 21 around the axis of the adjustment cylinder 21 on its wall. By configuring the air holes 21a, the adjustment cylinder 21 can more easily generate the extension/retraction amount along the predetermined direction OO’ under compressive force. This allows the distance between the outer end surface 10a of the mask body 10 and the inner surface of the head-wearable device to be adjusted according to different users' facial structures, ensuring that the mask body 10 can completely block light around the user's eyes while enhancing wearing comfort.

As shown in FIGS. 1 and 2, to further enhance adjustability of the adjustment cylinder 21 under compressive force, in further embodiments, the adjustment cylinder 21 includes a first cylinder segment 211 and a second cylinder segment 212 connected to an end of the first cylinder segment 211. The first cylinder segment 211 is adjacent to the outer end surface 10a of the mask body 10 and the second cylinder segment 212 is arranged on the end of the first cylinder segment 211 facing away from the outer end surface 10a. Diameters of the first cylinder segment 211 and the second cylinder segment 212 may include, but not limited to, the following embodiments.

In a first embodiment, from the side adjacent to the outer end surface 10a of the mask body 10 to the side farther away from the outer end surface 10a, the diameter of at least a portion of the first cylinder segment 211 gradually decreases, and the diameter of at least a portion of the second cylinder segment 212 gradually decreases. Furthermore, the diameter of the end of the first cylinder segment 211 facing away from the outer end surface 10a is less than or equal to the diameter of the end of the second cylinder segment 212 adjacent to first cylinder segment 211. This results in the adjustment cylinder 21 having a larger diameter at the end adjacent to the outer end surface 10a of the mask body 10 and a smaller diameter at the end farther away from the outer end surface 10a. The adjustment cylinder 21 is in a conical structure. This configuration allows the adjustment cylinder 21 to exhibit excellent adjustability under compressive force, which is beneficial for better adjustment of the distance between the mask body 10 and the inner surface of the head-wearable device according to different users' facial structures. It ensures that the mask body 10 can completely block light around the user's eyes while improving wearing comfort.

In a second embodiment, from the side adjacent to the outer end surface 10a of the mask body 10 to the side farther away from the outer end surface 10a, the diameter of at least a portion of the first cylinder segment 211 gradually increases, and the diameter of at least a portion of the second cylinder segment 212 gradually increases. Additionally, the diameter of the end of the first cylinder segment 211 facing away from the outer end surface 10a is greater than or equal to the diameter of the end of the second cylinder segment 212 adjacent to the first cylinder segment 211. It results in the adjustment cylinder 21 having a smaller diameter at the end adjacent to the outer end surface 10a of the mask body 10 and a larger diameter at the end farther away from the outer end surface 10a. The adjustment cylinder 21 is in an inverted conical structure. This configuration allows the adjustment cylinder 21 to exhibit excellent adjustability under compressive force, which is beneficial for better adjustment of the distance between the mask body 10 and the inner surface of the head-wearable device according to different users' facial structures. It ensures that the mask body 10 can completely block light around the user's eyes while improving wearing comfort.

It should be noted that the first cylinder segment 211 and the second cylinder segment 212 may be, but not limited to, formed as an integrated structure through methods such as injection molding or 3D printing. The difference in diameter at a junction between the first cylinder segment 211 and the second cylinder segment 212 allows this connection area to more easily generate an extension/retraction amount under compressive force. Furthermore, the adjustment cylinder 21 in the embodiments of the present disclosure is not limited to including only the first cylinder segment 211 and the second cylinder segment 212. It may further include a third cylinder segment (not shown in the figures) connected to the end of the second cylinder segment 212 facing away from the first cylinder segment 211, a fourth cylinder segment (not shown in the figures) connected to the end of the third cylinder segment facing away from the second cylinder segment 212, and so on. Reasonable configuration can be made according to actual needs.

In the third embodiment, from the side adjacent to the outer end surface 10a of the mask body 10 to the side farther away from the outer end surface 10a, the diameter of at least a portion of the first cylinder segment 211 gradually decreases, while the diameter of at least a portion of the second cylinder segment 212 gradually increases. In this case, the adjustment cylinder 21 overall is shaped as an hourglass-like structure.

As shown in FIGS. 2, 4 to 6, the adjustment member 20 further includes a connection body 22 connected to the end of the adjustment cylinder 21 facing away from the outer end surface 10a of the mask body 10. The eye mask 1 has a first usage state. In the first usage state, the connection body 22 serves as the second end of the adjustment member 20 and is used for connection with a first lens 511 of a first pair of eyeglasses 51.

The connection body 22 may be integrally formed with the adjustment cylinder 21 through methods such as injection molding or 3D printing, but is not limited to these methods.

The head-wearable device includes the first pair of eyeglasses 51. It should be noted that the first pair of eyeglasses 51 is an embodiment of the head-wearable device. This first pair of eyeglasses 51 includes the first lens 511, and the connection body 22 is connected to the first lens 511 to position the eye mask 1 on the first lens 511. The specific connection method between the connection body 22 and the first lens 511 is not limited here, and designers can make reasonable configuration according to actual needs. For example, the connection body 22 may be removably connected to the first lens 511 through at least one method such as snapping or plugging. Alternatively, the connection body 22 may be non-removably connected to the first lens 511 through methods such as bonding, injection molding, or 3D printing.

The eye mask 1 has a first usage state. In the first usage state, the connection body 22 is directly connected to the first lens 511 of the first pair of eyeglasses 51 to position the eye mask 1 on the first lens 511. When the user wears the first pair of eyeglasses 51, the eye mask 1 covers the user's eyes accordingly, providing an excellent shading effect.

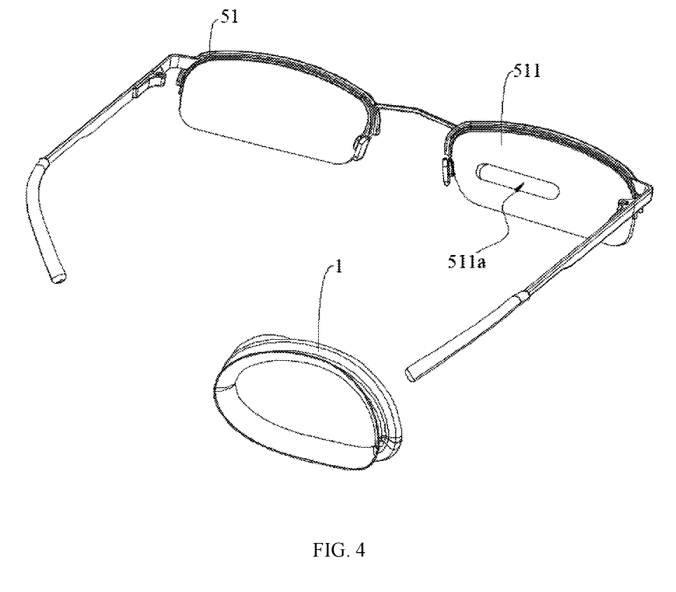

Furthermore, as shown in FIGS. 4 to 6, in the first usage state, the position of the connection body 22 relative to the first lens 511 is adjustable along a transverse direction of the first pair of eyeglasses 51. By adjusting the position of the connection body 22, the relative position between the eye mask 1 and the first lens 511 can be changed, making it suitable for users with different interpupillary distances and enhancing the applicability of the eye mask 1.

Specifically, an inner surface of the first lens 511 has a sliding groove 511a extending along the transverse direction of the first pair of eyeglasses 51. The connection body 22 is slidably connected to the sliding groove 511a, which allows the eye mask 1 to be positionally adjustable relative to the first lens 511 in the first usage state. By configuring the connection body 22 to be slidably connected to the sliding groove 511a of the first lens 511, the entire eye mask 1 can slide a certain distance relative to the first lens 511 along the transverse direction of the first pair of eyeglasses 51. This allows different users to slide the eye mask 1 to a suitable position relative to the first lens 511 according to their actual needs, enabling the eye mask 1 to be worn by users with different face shapes, thereby enhancing its applicability. It is worth mentioning that the eye mask 1 can slide a certain distance relative to the first lens 511 along the transverse direction of the first pair of eyeglasses 51 via the connection body 22. This allows users to slide the eye mask 1 to an appropriate position relative to the first lens 511 according to their actual needs, thereby optimizing the shading effect of the eye mask 1.

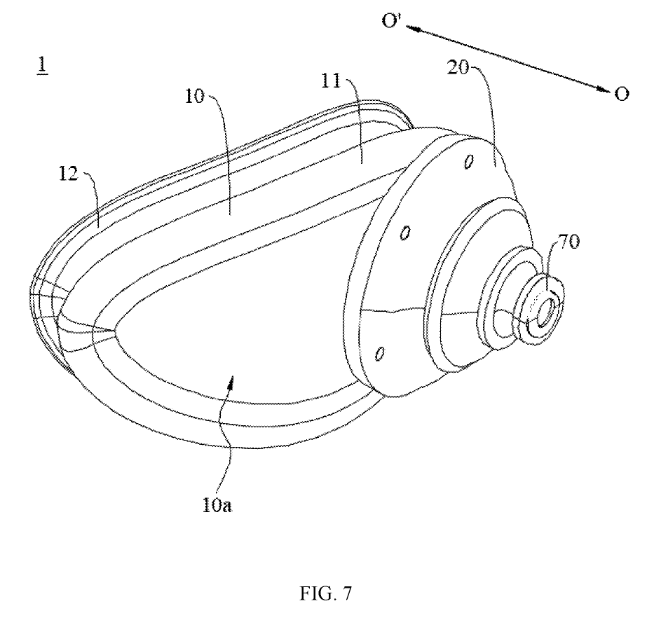

More specifically, the connection body 22 includes a first connection portion 221 and a second connection portion 222. The first connection portion 221 is connected to an end of the adjustment cylinder 21 facing away from the outer end surface 10a of the mask body 10. The second connection portion 222 is connected to an end of the first connection portion 221 facing away from the adjustment cylinder 21. In the first usage state, at least part of the second connection portion 222 is embedded in the sliding groove 511a of the first lens 511. As shown in FIG. 7, the eye mask 1 further includes a reinforcement piece 70 arranged on the second connection portion 222. The first connection portion 221 and the second connection portion 222 are integrally formed so that a cross-section of the connection body 22 is in a T-shape. The reinforcement piece 70 may be a stainless-steel sheet embedded in the second connection portion 222, or it may be an iron sheet embedded in the second connection portion 222. By configuring the reinforcement piece 70, it can enhance a structural strength of the second connection portion 222, thereby improving connection stability between the connection body 22 and the first lens 511, thus enhancing connection stability between the eye mask 1 and the first lens 511.

In the first usage state, the connection body 22 can rotate relative to the first lens 511. It allows to adjust a relative angle between the eye mask 1 and the first lens 511 by rotating the connection body 22, which is suitable for users with different face shapes and enhancing the applicability of the eye mask 1.

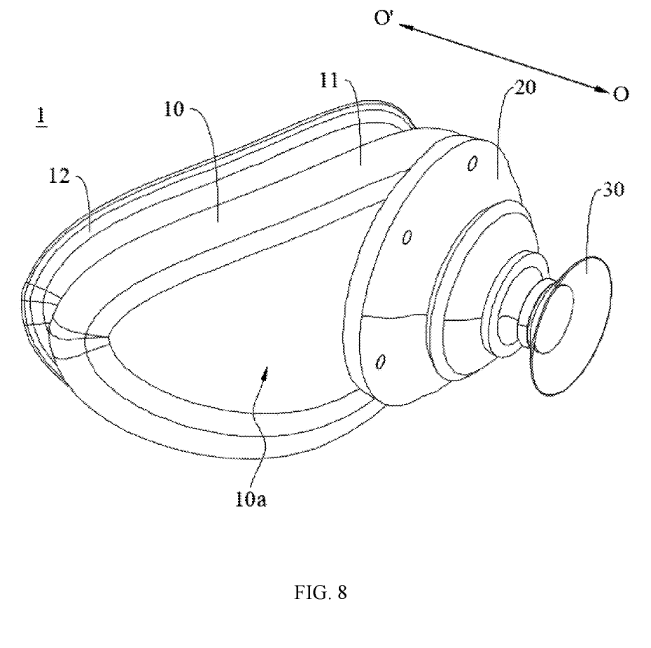

As shown in FIGS. 8 to 12, the eye mask 1 further includes a suction cup 30 connected to the connection body 22. The eye mask 1 further has a second usage state, in which the connection body 22 serves as the second end of the adjustment member 20 and is detachably connected to a second lens 521 of a second pair of eyeglasses 52 via the suction cup 30.

The head-wearable device further includes the second pair of eyeglasses 52. It should be noted that the second pair of eyeglasses 52 is another embodiment of the head-wearable device, different from the first pair of eyeglasses 51. This second pair of eyeglasses 52 includes the second lens 521, and the connection body 22 is connected to the second lens 521 indirectly through the suction cup 30 to position the eye mask 1 on the second lens 521. It can be understood that the main structural difference between the second pair of eyeglasses 52 and the first pair of eyeglasses 51 lies in the second lens 521 versus the first lens 511. Specifically, the inner surface of the first lens 511 is configured with structures (such as the aforementioned sliding groove 511a) for direct connection with the connection body 22, whereas the inner surface of the second lens 521, like that of ordinary lenses, is smooth and flat.

The specific connection method between the suction cup 30 and the connection body 22 is not limited here, and designers may make reasonable configuration according to actual needs. For example, the suction cup 30 may be detachably connected to the connection body 22 through at least one method such as screwing, snapping, or plugging. Alternatively, the suction cup 30 may be non-removably connected to the connection body 22 through methods such as bonding, injection molding, or 3D printing.

The eye mask 1 further has a second usage state. In the second usage state, the connection body 22 is connected to the second lens 521 of the second pair of eyeglasses 52 indirectly through the suction cup 30, positioning the eye mask 1 on the second lens 521. When the user wears the second pair of eyeglasses 52, the eye mask 1 covers the user's eyes accordingly, providing an effective shading effect.

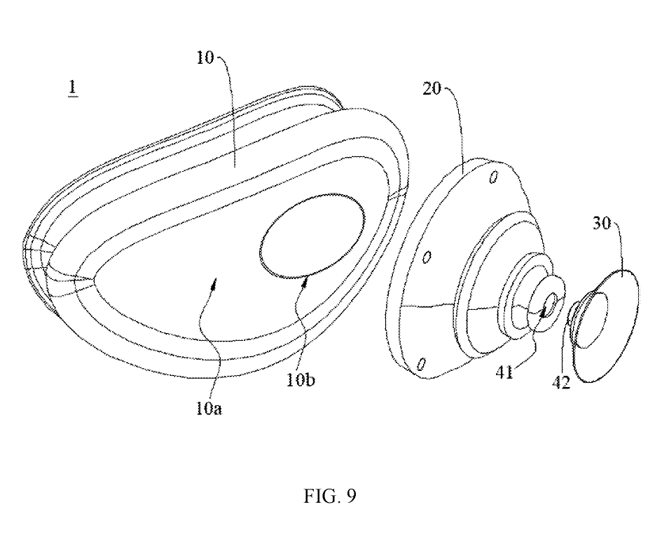

Furthermore, as shown in FIGS. 8 and 9, one of the suction cup 30 and the connection body 22 is provided with a protrusion 42, and another of the suction cup 30 and the connection body 22 is provided with a recess 41. The protrusion 42 engages with the recess 41 to position the suction cup 30 relative to the connection body 22. The protrusion 42 may form an integrated structure with the one of the suction cup 30 and the connection body 22 through methods such as injection molding or 3D printing, but is not limited to these methods. Similarly, the recess 41 may be integrally formed on the another of the suction cup 30 and the connection body 22 through methods such as injection molding or 3D printing. By configuring the protrusion 42 and the recess 41, with the protrusion 42 inserted into the recess 41, an interference fit is formed between the outer surface of the protrusion 42 and the wall surface of the recess 41. This achieves relative positional fixation between the suction cup 30 and the connection body 22. The structure is simple, making it convenient for users to install and remove the suction cup 30.

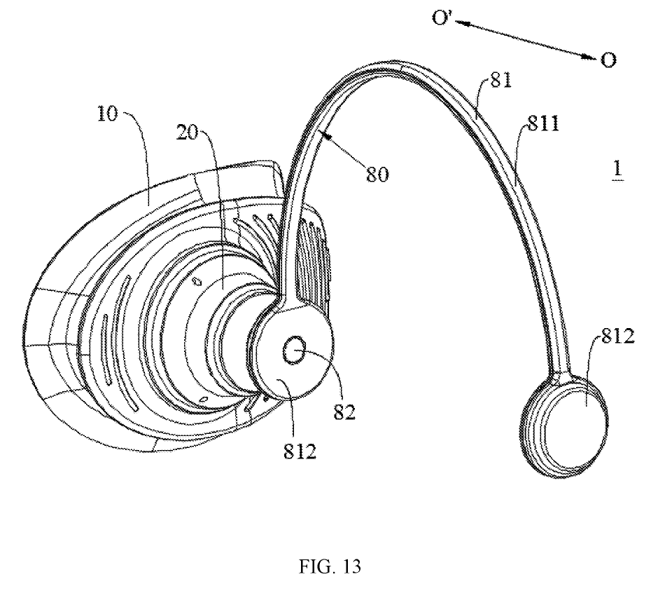

As shown in FIGS. 13 to 18, the eye mask 1 further includes a magnetic attraction structure 80 connected to the connection body 22. The eye mask 1 further has a third usage state, in which the connection body 22 serves as the second end of the adjustment member 20 and is positioned on a third lens 561 of a third pair of eyeglasses 56 via the magnetic attraction structure 80.

The head-wearable device includes the third pair of eyeglasses 56. It should be noted that the third pair of eyeglasses 56 is another embodiment of the aforementioned head-wearable device. The specific structure of the third pair of eyeglasses 56 may be the same as or different from that of the first pair of eyeglasses 51. The third pair of eyeglasses 56 includes the third lens 561, and the magnetic attraction structure 80 is directly connected to the third lens 561 via magnetic attraction to position the eye mask 1 on the third lens 561.

The specific connection method between the magnetic attraction structure 80 and the connection body 22 is not limited here, and designers may make reasonable configuration according to actual needs. For example, the magnetic attraction structure 80 may be detachably connected to the connection body 22 through at least one method such as screwing, snapping, or plugging, but is not limited to these methods.

The eye mask 1 further has a third usage state. In the third usage state, the connection body 22 is connected to the third lens 561 of the third pair of eyeglasses 56 indirectly through the magnetic attraction structure 80, thus positioning the eye mask 1 on the third lens 561. When the user wears the third pair of eyeglasses 56, the eye mask 1 covers the user's eyes accordingly, providing an effective shading effect.

The magnetic attraction structure 80 includes a connecting member 81 and two magnets 82. The connecting member 81 is elongated, with its connecting end attached to the connection body 22 and its free end suspended. One of the two magnets 82 is located at the connecting end of the connecting member 81, and the other magnet 82 is located at the free end. The magnets 82 may be connected to the connecting end (and free end) of the connecting member 81 through at least one method such as screwing, snapping, or plugging, but are not limited to these methods. The elongated connecting member 81 serves to integrate the two magnets 82 as a single unit. The magnet 82 on the connecting end is configured to adhere to an inner surface of the third lens 561 of the third pair of eyeglasses 56, while the magnet 82 on the free end is configured to adhere to an outer surface of the third lens 561. The two magnets 82 are aligned, with opposite polarities attracting each other, thereby achieving relative positional fixation between the eye mask 1 and the third lens 561 of the third pair of eyeglasses 56. It should be noted that the relative position between the eye mask 1 and the third lens 561 can be adjusted by moving the two magnets 82 along the third lens 561 to accommodate the wearing needs of users with different face shapes, making adjustments more convenient.

The connecting member 81 includes a connecting cord 811 and two connecting bases 812 connected to two opposite ends of the connecting cord 811 respectively. One connecting base 812 serves as the connecting end of the connecting member 81 and is detachably connected to the connection body 22, while the other connecting base 812 serves as the free end of the connecting member 81. Both two magnets 82 are at least partially embedded in corresponding connecting bases 812. The connecting cord 811 may be, but not limited to, a flexible rubber cord or a bendable rigid chain cord. The connecting base 812 connected to the connection body 22 is provided with one of a catch 91 and a slot 92, while the connection body 22 is provided with the other of the catch 91 and the slot 92. The catch 91 engages with the slot 92 to achieve a detachable connection between the connecting base 812 and the connection body 22, which is simple in structure and easy for manufacture and assembly.

In at least one embodiment, the magnetic attraction structure 80 may include a connecting member 81, a magnet 82, and a steel body 83. The connecting member 81 is elongated, with its connecting end attached to the connection body 22 and its free end suspended. One of the magnet 82 and the steel body 83 is located at the connecting end of the connecting member 81, and the other is located at the free end. The one of the magnet 82 and the steel body 83 may be connected to the connecting end of the connecting member 81 through at least one method such as screwing, snapping, or plugging; while the other of the magnet 82 and the steel body 83 may be connected to the free end through similar methods, though not limited to these methods. The elongated connecting member 81 serves to integrate the magnet 82 and the steel body 83 as a single unit. The magnet 82 (or steel body 83) on the connecting end is configured to adhere to the inner surface of the third lens 561 of the third pair of eyeglasses 56, while the steel body 83 (or magnet 82) on the free end is configured to adhere to the outer surface of the third lens 561. The magnet 82 and steel body 83 are aligned, with the magnet 82 attracting the steel body 83, thereby achieving relative positional fixation between the eye mask 1 and the third lens 561 of the third pair of eyeglasses 56. It should be noted that the relative position between the eye mask 1 and the third lens 561 can be adjusted by moving the magnet 82 and steel body 83 along the third lens 561 to fulfil wearing needs of users with different face shapes, making adjustments more convenient.

It should be noted that when the suction cup 30 is detached from the connection body 22, the eye mask 1 can be slidably connected to the first sliding groove 511a on the first lens 511 of the first pair of eyeglasses 51 via the connection body 22, to make the eye mask 1 in the first usage state. When the suction cup 30 is assembled onto the connection body 22, the eye mask 1 adheres to the second lens 521 of the second pair of eyeglasses 52 via the suction cup 30, to have the eye mask 1 in the second usage state. When the magnetic attraction structure 80 is assembled onto the connection body 22, the eye mask 1 is magnetically attached to the third lens 561 of the third pair of eyeglasses 56 via the magnetic attraction structure 80, to have the eye mask 1 in the third usage state. The eye mask 1 in the embodiments of the present disclosure has three usage states (namely, the aforementioned first, second, and third usage states). Users can assemble the eye mask 1 onto the corresponding eyeglass lens according to their actual needs to achieve different usage states. Compared to the eye masks in related technologies, which only has a single usage state, the eye mask 1 in the embodiments of the present disclosure has a wider range of applicability.

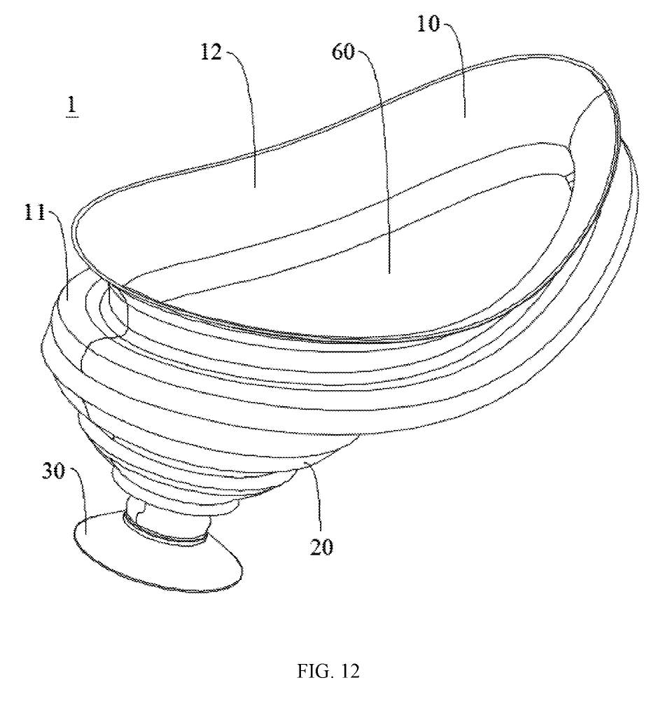

As shown in FIGS. 9 and 12, the outer end surface 10a of the mask body 10 is provided with a through-hole 10b communicated with the adjustment cylinder 21. The edge of the through-hole 10b is spaced apart from an outer edge of the outer end surface 10a. The eye mask 1 further includes a gasket 60 arranged inside the mask body 10 to cover the through-hole 10b. The material of the gasket 60 may include, but not limited to, flexible materials such as sponge. By configuring the gasket 60, it not only enhances the overall shading effect of the eye mask 1 but also provides waterproofing and dustproofing, while offering cushioning protection for the user's eyes and surrounding skin.

Considering that the adjustment member 20 can exhibit good adjustability under compressive force, the material for making the adjustment member 20 may include, but not limited to, flexible materials such as elastic rubber, elastic silicone, or elastic resin. Of course, the manufacturing material for the adjustment member 20 may further include, but not limited to, rigid materials such as plastic.

As shown in FIG. 12, the mask body 10 includes a mask frame 11 and a skirt 12. The mask frame 11 includes the outer end surface 10a. At least a portion of the skirt 12 is located on the side of the mask frame 11 opposite to the outer end surface 10a. The skirt 12 is connected to the mask frame 11 and is arranged in an annular shape along the circumference of the mask frame 11. The skirt 12 may be integrally formed with the mask frame 11 through methods such as injection molding or 3D printing, but is not limited to these methods. By configuring the mask frame 11, the mask frame 11 serves as the main carrier of the mask body 10, providing support for components such as the adjustment member 20. By configuring the skirt 12, which annularly surrounds the circumference of the mask frame 11, when the eye mask 1 is in a worn state, the skirt 12 conforms to the skin around the user's eyes, achieving complete shading around the ocular area.

Furthermore, the skirt 12 is symmetrically configured in the vertical direction (i.e., the upper edge and lower edge of the skirt 12 are symmetrical) perpendicular to the transverse direction of the eye mask. Taking a head-wearable device that includes eyeglasses with dual lenses as an example, the skirt 12 is symmetrically arranged relative to a transverse direction of the eyeglasses. It allows an eye mask 1 fixed on one lens of the eyeglasses to be flipped 180 degrees about a longitudinal direction of the eyeglasses and then fixed on the other lens of the eyeglasses, which makes the eye mask 1 suitable for both the left and right eyes, thereby enhancing its applicability. It should be noted that an outer lateral flange of the skirt 12 corresponding to the user's outer eye corner is longer than an inner lateral flange corresponding to the user’s inner eye corner. This effectively reduces likelihood of light leakage from the outer eye corner.

Considering that the mask body 10 directly contacts the skin around the user's eyes, to enhance the wearing comfort of the eye mask 1, the manufacturing material of the mask body 10 may include, but not limited to, flexible materials such as elastic rubber, elastic silicone, or elastic resin.

As shown in FIGS. 1 and 8, the adjustment member 20 and the mask body 10 are integrally formed. The adjustment member 20 may be integrally formed with the mask body 10 through methods such as injection molding or 3D printing, but is not limited to these methods. This effectively reduces a manufacturing difficulty between the adjustment member 20 and the mask body 10.

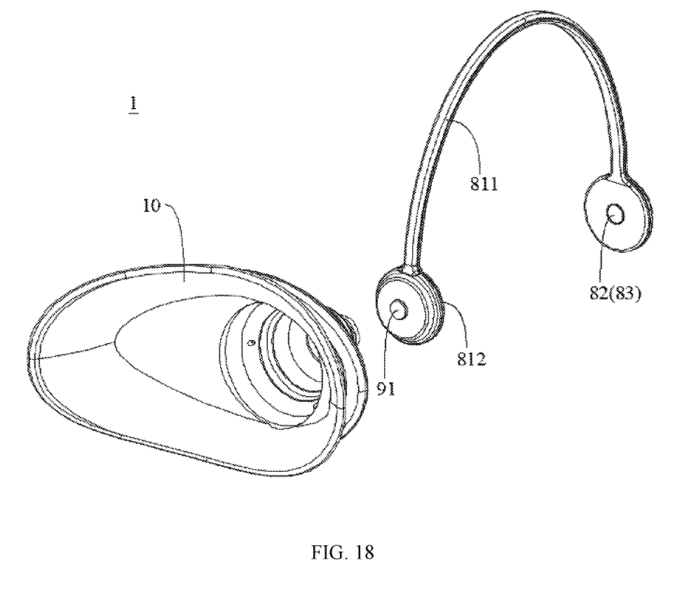

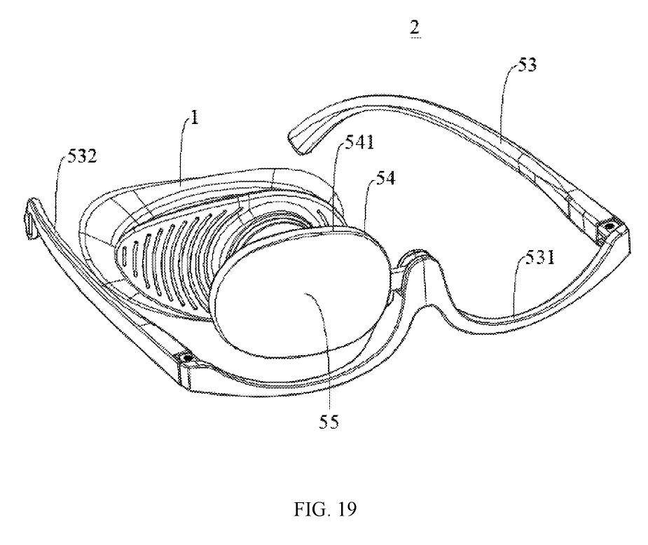

Referring to FIGS. 18 and 19, in a second aspect, an embodiment of the present disclosure provides an eye mask set 2 including the head-wearable device and the eye mask 1. The eye mask 1 is connected to the head-wearable device.

Based on the eye mask set 2 in the embodiments of the present disclosure, which includes the eye mask 1, when the head-wearable device is in a worn state, the eye mask 1 covers the user's eyes. The adjustment member 20 can be subjected to compressive forces from different users' faces, generating varying extension/retraction amounts along the predetermined direction OO’. This allows the distance between the outer end surface 10a of the mask body 10 and the inner surface of the head-wearable device to be optimized for different users. Consequently, it effectively ensures that the mask body 10 can completely block light around the eyes of various users, thereby enhancing the applicability of the eye mask 1.

Furthermore, the head-wearable device includes a frame 53 and a rotating member 54. The frame 53 is configured to be worn on the user's head, and the rotating member 54 is rotatably connected to the frame 53. The second end of the adjustment member 20 facing away from the outer end surface 10a of the mask body 10 is connected to the rotating member 54. The frame 53 serves as a structure worn on the user's head. The eye mask 1 can be switched from in front of one eye to in front of the other eye by rotating of the rotating member 54, thereby making the eye mask 1 suitable for both the left and right eyes.

Specifically, as shown in FIG. 19, the rotating member 54 includes a rotating frame 541 rotatably connected to the frame 53. The head-wearable device further includes a lens 55 installed on the rotating frame 541. The second end of the adjustment member 20 facing away from the outer end surface 10a of the mask body 10 is connected to the lens 55. It should be noted that the frame 53, rotating frame 541, and lens 55 cooperate to form a "single-lens" style eyeglass as the head-wearable device. When the inner surface of the lens 55 is provided with a sliding groove 511a, the eye mask 1 is in the first usage state, where it is slidably connected to the sliding groove 511a on the lens 55 via the connection body 22. When the inner surface of the lens 55 is not provided with the sliding groove 511a, the eye mask 1 is in the second usage state, where it adheres to the lens 55 via the suction cup 30.

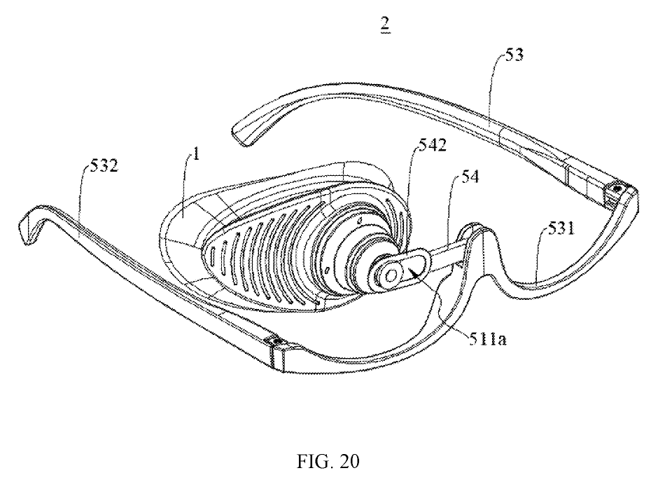

Specifically, as shown in FIG. 20, the rotating member 54 includes a rotating rod 542 rotatably connected to the frame 53. The inner surface of the rotating rod 542 is provided with a sliding groove 511a. The second end of the adjustment member 20 away from the outer end surface 10a of the mask body 10 is connected to the rotating rod 542. It should be noted that the inner surface of the rotating rod 542 is provided with the sliding groove 511a, which makes the eye mask 1 in the first usage state where it is connected to the sliding groove 511a on the rotating rod 542 via the connection body 22.

Specifically, as shown in FIGS. 19-20, the frame 53 includes a bridge frame 531 and two support legs 532. The two support legs 532 are respectively connected to two opposite ends of the bridge frame 531. When the frame 53 is worn on the user's head, the two support legs 532 rest on user's ears, and the bridge frame 531 rests on the user's nose bridge. The rotating member 54 is rotatably connected to a central portion of the bridge frame 531.

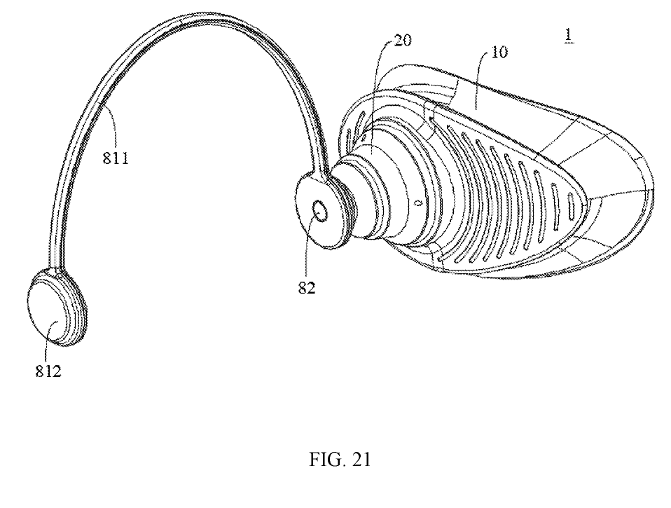

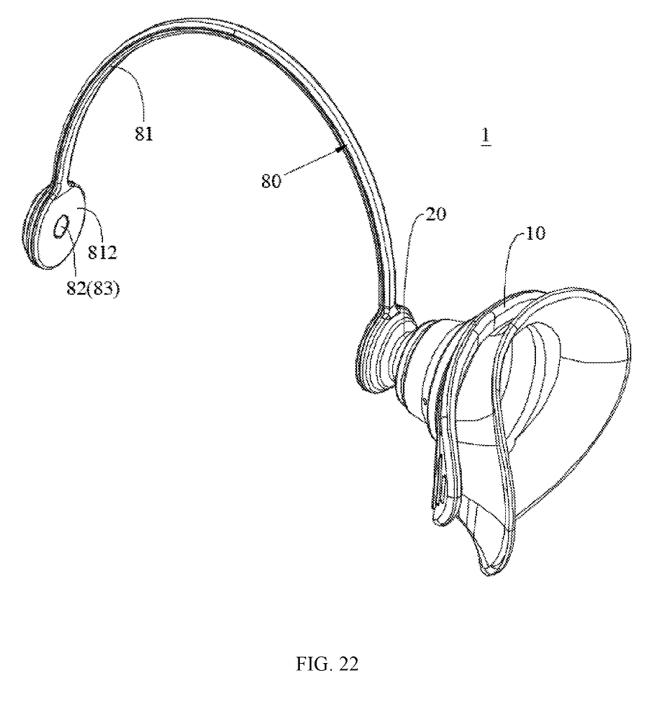

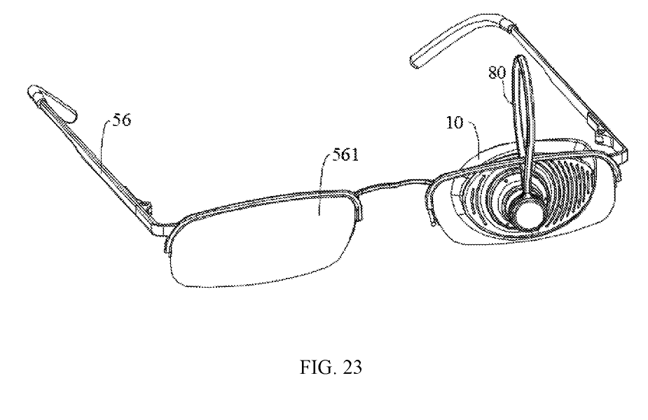

Referring to FIGS. 21-23, in a third aspect, an embodiment of the present disclosure provides an eye mask 1, which is used for connection with a head-wearable device. The eye mask 1 includes a mask body 10, an adjustment member 20, and a magnetic attraction structure 80. The mask body 10 has an outer end surface 10a. At least a portion of the adjustment member 20 is located on a side of the outer end surface 10a of the mask body 10, and a first end of the adjustment member 20 adjacent to the outer end surface 10a is connected to the mask body 10. The magnetic attraction structure 80 is connected to a second end of the adjustment member 20 facing away from the outer end surface 10a. The magnetic attraction structure 80 is configured to be connected to the head-wearable device via magnetic attraction to position the eye mask 1 on the head-wearable device.

The configuration of the mask body 10 can be referred to the aforementioned embodiments and will not be repeated here.

Based on the eye mask 1 in the embodiments of the present disclosure, by configuring the magnetic attraction structure 80, the second end of the adjustment member 20 facing away from the outer end surface 10a of the mask body 10 is connected to the head-wearable device via the magnetic attraction structure 80, positioning the eye mask 1 on the head-wearable device. When the user wears the head-wearable device, the eye mask 1 covers the user's eyes accordingly, providing an effective shading effect.

The adjustment member 20 is configured to generate an extension/retraction amount along a predetermined direction OO’ when subjected to force, thereby allowing a distance between the outer end surface 10a of the mask body 10 and the inner surface of the head-wearable device to be adjustable. The predetermined direction OO’ is inclined relative to the outer end surface 10a of the mask body 10. The configuration of the adjustment member 20 can be referred to the aforementioned embodiments and will not be repeated here. By configuring the adjustment member 20 to generate the extension/retraction amount along the predetermined direction OO’ when subjected to force, the distance between the outer end surface 10a of the mask body 10 and the inner surface of the head-wearable device can be adjusted. Thus, when the eye mask 1 is in a worn state, the adjustment member 20 can be subjected to compressive forces from different users' faces, generating varying extension/retraction amounts along the predetermined direction OO’. This allows the distance between the outer end surface 10a of the mask body 10 and the inner surface of the head-wearable device to be optimized for different users, effectively ensuring that the mask body 10 can completely block light around the eyes of various users. This enhances the applicability of the eye mask 1 while improving user comfort during wear.

As shown in FIGS. 21 to 23, the magnetic attraction structure 80 includes a connecting member 81 and two magnets 82. The connecting member 81 is elongated, with its connecting end attached to the second end of the adjustment member 20 and its free end suspended. One magnet 82 is located at the connecting end of the connecting member 81, and the other magnet 82 is located at the free end of the connecting member 81. The magnets 82 may be connected to the connecting end (and free end) of the connecting member 81 through at least one method such as screwing, snapping, or plugging, but are not limited to these methods. The elongated connecting member 81 serves to integrate the two magnets 82 as a single unit. The magnet 82 on the connecting end is configured to adhere to the inner surface of the head-wearable device, while the magnet 82 on the free end is configured to adhere to the outer surface of the head-wearable device. The two magnets 82 are aligned, with opposite polarities attracting each other, thereby achieving relative positional fixation between the eye mask 1 and the head-wearable device. It should be noted that the relative position between the eye mask 1 and the head-wearable device can be adjusted by moving the two magnets 82 to better fit the wearing needs of users with different face shapes, making adjustments more convenient.

The connecting member 81 includes a connecting cord 811 and two connecting bases 812 connected two opposite ends of the connecting cord 811 respectively. One connecting base 812 serves as the connecting end of the connecting member 81 and is non-removably connected to the second end of the adjustment member 20, while the other connecting base 812 serves as the free end of the connecting member 81. Each of the two magnets 82 is at least partially embedded in corresponding connecting base 812. The connecting cord 811 may be, but not limited to, a flexible rubber cord or a bendable rigid chain cord.

The connecting base 812 is integrally formed with the second end of the adjustment member 20. The connecting base 812 may be integrally formed with the second end of the adjustment member 20 through methods such as injection molding or 3D printing, but is not limited to these methods.

In at least one embodiment, the magnetic attraction structure 80 may include a connecting member 81, a magnet 82, and a steel body 83. The connecting member 81 is elongated, with its connecting end attached to the second end of the adjustment member 20 and its free end suspended. One of the magnet 82 and the steel body 83 is arranged at the connecting end of the connecting member 81, and the other of the magnet 82 and the steel body 83 is arranged at the free end. The one of the magnet 82 and the steel body 83 may be connected to the connecting end of the connecting member 81 through at least one method such as screwing, snapping, or plugging; while the other of the magnet 82 and the steel body 83 may be connected to the free end through similar methods, but not limited to these methods. The elongated connecting member 81 serves to integrate the magnet 82 and the steel body 83 as a single unit. The magnet 82 (or the steel body 83) on the connecting end is configured to adhere to the inner surface of the head-wearable device, while the steel body 83 (or magnet 82) on the free end is configured to adhere to the outer surface of the head-wearable device. The magnet 82 and steel body 83 are aligned, with the magnet 82 attracting the steel body 83, thereby achieving relative positional fixation between the eye mask 1 and the head-wearable device. It should be noted that the relative position between the eye mask 1 and the head-wearable device can be adjusted by moving the magnet 82 and steel body 83 to better match the wearing needs of users with different face shapes, making adjustments more convenient.

In the drawings of the embodiments, identical or similar reference numerals correspond to identical or similar components. In the description of the present disclosure, it should be understood that orientation or positional relationships indicated by terms such as "upper," "lower," "left," and "right" are based on the orientation or positional relationships shown in the drawings. These terms are used solely for convenience of describing present disclosure and simplifying the description, and do not indicate or imply that the referred devices or elements must have a specific orientation or be constructed and operated in a specific orientation. Therefore, the terms describing positional relationships in the drawings are for illustrative purposes only and shall not be construed as limiting this application. For those skilled in the art, the specific meanings of the aforementioned terms may be understood according to specific circumstances.

The above descriptions are merely preferred embodiments of present disclosure and are not intended to limit this application. Any modifications, equivalent substitutions, and improvements made within the spirit and principles of this application shall be included within the scope of protection of this application.

What is claimed is:

1. An eye mask, configured to be connected to a head-wearable device, wherein the eye mask comprises:
a mask body, having an outer end surface; and
an adjustment member, wherein at least a portion of the adjustment member is arranged on a side of the outer end surface of the mask body; a first end of the adjustment member adjacent to the outer end surface of the mask body is connected to the mask body, and a second end of the adjustment member facing away from the outer end surface is configured to be connected to the head-wearable device; the adjustment member is configured to generate an extension/retraction amount along a predetermined direction OO’ when subjected to force, thereby allowing a distance between the outer end surface of the mask body and an inner surface of the head-wearable device to be adjustable; and the predetermined direction OO’ is inclined relative to the outer end surface of the mask body.

2. The eye mask according to claim 1, wherein the adjustment member comprises an adjustment cylinder configured to generate the extension/retraction amount along the predetermined direction OO’; an end of the adjustment cylinder adjacent to the outer end surface of the mask body serves as the first end of the adjustment member and is connected to the mask body;
wherein,
from a side adjacent to the outer end surface of the mask body to a side farther away from the outer end surface, a diameter of at least a portion of the adjustment cylinder gradually decreases; or
from a side adjacent to the outer end surface of the mask body to a side farther away from the outer end surface, a diameter of at least a portion of the adjustment cylinder gradually increases.

3. The eye mask according to claim 2, wherein the adjustment cylinder comprises a first cylinder segment and a second cylinder segment connected to an end of the first cylinder segment; the first cylinder segment is adjacent to the outer end surface of the mask body and the second cylinder segment is arranged on the end of the first cylinder segment facing away from the outer end surface;
from the side adjacent to the outer end surface of the mask body to the side farther away from the outer end surface, a diameter of at least a portion of the first cylinder segment gradually decreases, and a diameter of at least a portion of the second cylinder segment gradually decreases; and a diameter of an end of the first cylinder segment facing away from the outer end surface is less than or equal to a diameter of an end of the second cylinder segment adjacent to first cylinder segment; or
from the side adjacent to the outer end surface of the mask body to the side farther away from the outer end surface, the diameter of at least a portion of the first cylinder segment gradually increases, and the diameter of at least a portion of the second cylinder segment gradually increases; and the diameter of the end of the first cylinder segment facing away from the outer end surface is less than or equal to the diameter of the end of the second cylinder segment adjacent to first cylinder segment.

4. The eye mask according to claim 2, wherein
the adjustment member further comprises a connection body connected to the end of the adjustment cylinder facing away from the outer end surface;
the eye mask has a first usage state where the connection body serves as the second end of the adjustment member and is configured to be connected a first lens of a first pair of eyeglasses; and the head-wearable device comprises the first pair of eyeglasses.

5. The eye mask according to claim 4, wherein
in the first usage state, a position of the connection body relative to the first lens is adjustable along a transverse direction of the first pair of eyeglasses; and/or
in the first usage state, the connection body is rotatable relative to the first lens.

6. The eye mask according to claim 5, wherein an inner surface of the first lens is provided with a sliding groove extending along the transverse direction of the first pair of eyeglasses; and
the connection body is slidably connected to the sliding groove to allow the position of the eye mask relative to the first lens to be adjustable in the first usage state.

7. The eye mask according to claim 6, wherein
the connection body comprises a first connection portion and a second connection portion; the first connection portion is connected to the end of the adjustment cylinder facing away from the outer end surface of the mask body, and the second connection portion is connected to an end of the first connection portion facing away from the adjustment cylinder; in the first usage state, the second connection portion is at least partially embedded in the sliding groove; and
the eye mask further comprises a reinforcement piece arranged on the second connection portion.

8. The eye mask according to claim 4, wherein
the eye mask further comprises a suction cup connected to the connection body;
the eye mask further has a second usage state where the connection body serves as the second end of the adjustment member and is detachably connected to a second lens of a second pair of eyeglasses via the suction cup; wherein the head-wearable device comprises the second pair of eyeglasses.

9. The eye mask according to claim 8, wherein one of the suction cup and the connection body is provided with a protrusion, and another of the suction cup and the connection body is provided with a recess configured to be engaged with the protrusion to position the suction cup relative to the connection body.

10. The eye mask according to claim 4, wherein
the eye mask further comprises a magnetic attraction structure connected to the connection body;
the eye mask further has a third usage state where the connection body serves as the second end of the adjustment member and is positioned on a third lens of a third pair of eyeglasses via the magnetic attraction structure; wherein the head-wearable device comprises the third pair of eyeglasses.

11. The eye mask according to claim 10, wherein
the magnetic attraction structure comprises a connecting member and two first magnets; the connecting member is elongated, a connecting end of the connecting member is attached to the connection body and a free end of the connecting member is suspended; one of the two first magnets is arranged at the connecting end of the connecting member, and another of the two first magnets is arranged at the free end of the connecting member; or
the magnetic attraction structure comprises the connecting member, and a second magnet, and a steel body; the connecting member is elongated, the connecting end of the connecting member is attached to the connection body and the free end of the connecting member is suspended; one of the second magnet and the steel body is arranged at the connecting end of the connecting member, and another of the second magnet and the steel body is arranged at the free end of the connecting member.

12. The eye mask according to claim 11, wherein
the connecting member comprises a connecting cord and two connecting bases connected to two opposite ends of the connecting cord respectively; one of the two connecting bases serves as the connecting end of the connecting member and is detachably connected to the connection body; and another of the two connecting bases serves as the free end of the connecting member; and each of the two magnets is at least partially embedded in a corresponding one of the two connecting bases.

13. The eye mask according to claim 1, wherein the mask body comprises
a mask frame, having the outer end surface; and
a skirt, at least partially arranged on a side of the mask frame facing away from the outer end surface; wherein the skirt is connected to the mask frame and is arranged in annular shape along a circumference of the mask frame; and the skirt is symmetrically configured in a vertical direction perpendicular to a transverse direction of the eye mask.

14. An eye mask set, comprising:
a head-wearable device; and
an eye mask, comprising a mask body and an adjustment member; wherein the mask body has an outer end surface, at least a portion of the adjustment member is arranged on a side of the outer end surface of the mask body; a first end of the adjustment member adjacent to the outer end surface of the mask body is connected to the mask body, and a second end of the adjustment member facing away from the outer end surface is configured to be connected to the head-wearable device; the adjustment member is configured to generate an extension/retraction amount along a predetermined direction OO’ when subjected to force, thereby allowing a distance between the outer end surface of the mask body and an inner surface of the head-wearable device to be adjustable; and the predetermined direction OO’ is inclined relative to the outer end surface of the mask body.

15. The eye mask set according to claim 14, wherein the head-wearable device comprises a frame and a rotating member; the frame is configured to be worn on the user's head, and the rotating member is rotatably connected to the frame 53; the second end of the adjustment member facing away from the outer end surface of the mask body is connected to the rotating member.

16. The eye mask set according to claim 15, wherein rotating member comprises a rotating frame rotatably connected to the frame; the head-wearable device further comprises a lens arranged on the rotating frame; and the second end of the adjustment member facing away from the outer end surface is connected to the lens.

17. An eye mask, configured to be connected to a head-wearable device, wherein the eye mask comprises:
a mask body, having an outer end surface;
an adjustment member, at least partially arranged on a side of the mask body where the outer end surface is located, a first end of the adjustment member adjacent to the outer end surface is connected to the mask body; and
a magnetic attraction structure, connected to a second end of the adjustment member facing away from the outer end surface; wherein the magnetic attraction structure is configured to be connected to the head-wearable device via magnetic attraction to position the eye mask on the head-wearable device.

18. The eye mask according to claim 17, wherein
the magnetic attraction structure comprises a connecting member and two first magnets; the connecting member is elongated, a connecting end of the connecting member is attached to the second end of the adjustment member, and a free end of the connecting member is suspended; one of the two first magnets is arranged at the connecting end of the connecting member, and another of the two first magnets is arranged at the free end of the connecting member; or
the magnetic attraction structure comprises the connecting member, and a second magnet, and a steel body; the connecting member is elongated, the connecting end of the connecting member is attached to the second end of the adjustment member, and the free end of the connecting member is suspended; one of the second magnet and the steel body is arranged at the connecting end of the connecting member, and another of the second magnet and the steel body is arranged at the free end of the connecting member.

19. The eye mask according to claim 18, wherein the connecting member comprises a connecting cord and two connecting bases connected to two opposite ends of the connecting cord respectively; one of the two connecting bases serves as the connecting end of the connecting member and is non-detachably connected to the second end of the adjustment member; and another of the two connecting bases serves as the free end of the connecting member; and each of the two magnets is at least partially embedded in a corresponding one of the two connecting bases.

20. The eye mask according to claim 19, wherein the one of the two connecting bases is integrally formed with the second end of the adjustment member.
